(19) United States
(12) Patent Application Publication
(10) Pub. No.: US 2012/0088944 A1
(43) Pub. Date: Apr. 12, 2012

(54) PROCESS FOR THE PREPARATION OF HYDROCARBONS

(75) Inventors: Andre BUIJS, Amsterdam (NL); Chippla Oliver VANDU, Amsterdam (NL)

(73) Assignee: SHELL OIL COMPANY, Houston, TX (US)

(21) Appl. No.: 13/267,948

(22) Filed: Oct. 7, 2011

(30) Foreign Application Priority Data
Oct. 11, 2010 (EP) 10187160.6

(51) Int. Cl. C07C 4/00

(52) U.S. Cl. 585/319

(57) ABSTRACT

The invention provides a process for the preparation of hydrocarbons comprising the steps of:
(a) contacting a mixture of carbon monoxide and hydrogen at an elevated temperature and pressure with a mixture of a methanol synthesis catalyst and a methanol conversion catalyst thereby forming C₅⁺ hydrocarbons;
(b) separating at least part of the C₅⁺ hydrocarbons as obtained in step (a) into a light stream and a heavy durene-rich stream;
(c) subjecting at least part of the heavy durene-rich stream to a hydrodealkylation treatment in the presence of hydrogen to obtain a stream of hydrocarbons having a reduced durene content; and
(d) mixing at least part of the light stream as obtained in step (b) with at least part of the stream of hydrocarbons having a reduced durene content as obtained in step (c).

This application claims the benefit of European Application No. 10187160.6 filed Oct. 11, 2010, which is incorporated herein by reference.

### FIELD OF THE INVENTION

The present invention relates to a process for the preparation of hydrocarbons, in particular gasolines and aromatic hydrocarbons.

### BACKGROUND OF THE INVENTION

Many documents are known describing methods and processes for the conversion of synthesis gas into hydrocarbons. The preparation of hydrocarbons from a H2/CO mixture by contacting this mixture at elevated temperature and pressure with a catalyst is known in the literature as the Fischer-Tropsch hydrocarbon synthesis. Catalysts often used for the purpose comprise one or more metals from the iron group, together with one or more promoters, and a carrier material. These catalysts can suitably be prepared by known techniques, such as precipitation, impregnation, kneading and melting. The products which can be prepared by using these catalysts generally have a very wide molecular weight distribution range. In addition to branched and unbranched paraffins, they often contain considerable amounts of olefins, oxygen-containing organic compounds and heavy aromatic hydrocarbons. Therefore, the direct conversion of H2/CO mixtures using the Fischer-Tropsch synthesis is not a very attractive route for the production of gasolines that meet environmental specifications.

### SUMMARY OF THE INVENTION

It has now been found that attractive gasoline components and aromatic hydrocarbons can be produced when use is made of a particular multi-step conversion process.

Accordingly, the present invention provides a process for the preparation of hydrocarbons comprising the steps of:
(a) contacting a mixture of carbon monoxide and hydrogen at an elevated temperature and pressure with a mixture of a methanol synthesis catalyst and a methanol conversion catalyst thereby forming C₅⁺ hydrocarbons;
(b) separating at least part of the C₅⁺ hydrocarbons as obtained in step (a) into a light stream and a heavy durene-rich stream;
(c) subjecting at least part of the heavy durene-rich stream to a hydrodealkylation treatment in the presence of hydrogen to obtain a stream of hydrocarbons having a reduced durene content; and
(d) mixing the at least part of the light stream as obtained in step (b) with at least part of the stream of hydrocarbons having a reduced durene content as obtained in step (c).
   An advantage of the present invention is that attractive gasoline components can be prepared, whereas at the same time valuable aromatic hydrocarbons can be produced for use as base chemicals for the production of a wide range of organic compounds. This combined production of gasoline components and aromatics is particularly attractive in view of the increasing demand for aromatics in the production of a wide variety of petrochemical compounds. This especially applies to benzene.

### DETAILED DESCRIPTION OF THE INVENTION

The invention provides a process for the preparation of hydrocarbons that can be used as gasoline components or as base chemicals in the chemical industry.

In step (a) a mixture of carbon monoxide and hydrogen is converted into a hydrocarbon mixture whose C₅⁺ fraction has a high content of branched C₅ and C₆ paraffins.

Suitably, step (a) is carried out at a temperature of 200-500° C., preferably in the range of from 300-450° C., a pressure in the range of from 1-150 bar, preferably in the range of from 5-100 bar, and a space velocity in the range of from 50-10000 N1.1-¹.h-¹, preferably in the range of from 300-3000 N1.1⁻¹.h⁻¹.

Suitably, unconverted synthesis gas can be recycled to step (a).

Preferably, the mixture of carbon monoxide and hydrogen in step (a) has a H₂/CO molar ratio in the range of from 0.3-2, more preferably in the range of from 0.4-1.

In step (a) use is made of a mixture of a methanol synthesis catalyst and a methanol conversion catalyst.

The methanol synthesis catalyst to be used in accordance with the present invention suitably comprises a zinc-containing composition which, in addition to zinc, comprises one or more metals chosen from chromium, copper and aluminium. The zinc-containing composition can suitably be prepared starting from one or more precipitates obtained by adding a basic reacting substance to one or more aqueous solutions containing salts of the metals involved.

Preferably, the methanol synthesis catalyst is a ZnO—Cr₂O₃ catalyst. Preferably, the atomic percentage of zinc calculated on the sum of zinc and chromium is 60-80%.

The methanol conversion catalyst to be used in accordance with the present invention suitably comprises a crystalline (metallo)silicate comprising in addition to SiO₂ one or more oxides of a trivalent metal chosen from aluminium, iron, gallium, rhodium, chromium and scandium, and having a SiO₂/Al₂O₃ molar ratio of at least 10.

Preferably, the methanol conversion catalyst is a crystalline iron alumina silicate or a zeolite selected from the group consisting of ZSM-5, ZSM-11, ZSM-12, ZSM-35 and ZSM-38. ZSM-5 is particularly preferred as zeolite.

The crystalline (metallo)silicate has suitably after one hour's calcination in air at 500° C., the following properties: (a) an X-ray powder diffraction pattern in which the strongest lines are the four lines mentioned in Table A,

TABLE A
d(Å)
11.1 +/− 0.2
10.0 +/− 0.2
3.84 +/− 0.07
3.72 +/− 0.06

In case the methanol conversion catalyst is a crystalline iron alumina silicate suitably the SiO₂/((Fe₂O₃+Al₂O₃) molar ratio is higher than 10, the SiO₂/Fe₂O₃ molar ratio is suitably lower than 250 and the SiO₂/Al₂O₃ molar ratio is suitably at least 50. The crystalline iron alumina silicate has preferably a SiO₂/Fe₂O₃ molar ratio higher than 25, but lower than 250 and a SiO₂/Al₂O₃ molar ratio of at least 50, but lower than 1200. More preferably, the crystalline iron alumina silicate has a SiO₂/Fe₂O₃ molar ratio of 50-175 and a SiO₂/Al₂O₃ molar ratio of at least 50, but lower than 800. In case the methanol conversion catalyst is a crystalline iron silicate, the SiO₂/Fe₂O₃ molar ratio is suitably higher than 25, but lower than 250. Preferably, the crystalline iron silicate has a SiO₂/Fe₂O₃ molar ratio in the range of from 50-175.

Suitably, the crystalline (metallo)silicate has an alkali metal content of less than 0.05% w.

In step (a) the weight ratio of the methanol synthesis catalyst to the methanol conversion catalyst is suitably in the range of from 0.1-12.5, and preferably in the range of from 0.5-10, and more preferably in the range of from 2.5-8,

The crystalline iron alumina silicates can be prepared starting from an aqueous mixture comprising the following compounds: one or more silicon compounds, one or more compounds which contain a monovalent organic cation (R) of from which such a cation is formed during the preparation of the silicate, one or more compounds in which iron and aluminium are present in trivalent form and one or more compounds of an alkali metal (M). The preparation is carried out by keeping the mixture at an elevated temperature until the silicate has formed, and subsequently separating the silicate crystals from the mother liquor and washing, drying and calcining the crystals. In the aqueous mixture from which the silicates are prepared the various compounds should be present in the following ratios, expressed in moles of the oxides:

### M₂O:SiO₂<0.35,

### R₂O:SiO₂=0.01-0.5,

### SiO₂:(Fe₂O₃+Al₂O₃)>10, and

### H₂O:SiO₂=5-100.

If in the preparation of the crystalline silicates the starting material is an aqueous mixture in which one or more alkali metal compounds are present, the crystalline silicates obtained will contain alkali metal. Depending on the concentration of alkali metal compounds in the aqueous mixture the crystalline silicates obtained may contain more than 1% w alkali metal. Since the presence of alkali metal in the crystalline silicates has an unfavourable influence on their catalytic properties, it is common practice in the case of crystalline silicates with a relatively high alkali metal content to reduce this content before using these silicates as catalysts. A reduction of the alkali metal content to less than 0.05% w is usually sufficient to this end. The reduction of the alkali metal content of crystalline silicates can very suitably be effected by treating the silicates once or several times with a solution of an ammonium compound. During this treatment alkali metal ions are exchanged for NH4⁺ ions and the silicate is converted to the NH4⁺ form. The NH4⁺ form of the silicate is converted to the H⁺ form by calcination.

In the preparation of the catalyst mixtures used in the present process use is made of one or more precipitates in which zinc occurs together with chromium and/or aluminium and which precipitates have been obtained by adding a basic reacting substance to one or more aqueous solution of salts of the metals involved. Preference is given to the use of precipitates in which, in addition to zinc, chromium occurs, in particular precipitates in which the atomic percentage of zinc, calculated on the sum of zinc and chromium, is at least 60% and more specifically 60-80%. The metal-containing precipitates may be prepared by precipitation of each of the metals individually or by co-precipitation of the desired metal combination. Preference is given to the use of a co-precipitate obtained by adding a basic reacting substance to an aqueous solution containing all the metals involved. This co-precipitation is preferably carried out in a mixing unit with a continuous supply of an aqueous solution containing the metal salts involved and an aqueous solution of the basic reacting substance in a stoichiometric quantity calculated on the metals, and with a continuous discharge of the co-precipitate formed.

The preparation of the catalyst mixtures used in the present process can be carried out in various ways. The precipitate may be calcined and then mechanically mixed with the crystalline silicate. The catalyst mixture may also very suitably be prepared by spray-drying. To this end the crystalline silicate is dispersed in water together with the precipitate mentioned hereinbefore, the dispersion thus obtained is spray-dried, and the spray-dried material is calcined. Spray-drying is a method used on a commercial scale for many years past for the preparation of small spherical particles from a solid material or a mixture of solids. The process is carried out by atomizing a dispersion in water of the material to be spray-dried through a nozzle or from a rotating disc into a hot gas. The process is particularly suitable for achieving intimate contact between different materials. In view of the form, size and strength of the catalyst particles prepared by spray-drying they are very suitable for use in a fluidized state.

In step (b) the separation will suitably be carried out at a temperature in the range of from 150-190° C. Preferably, the separation in step (b) is carried out at a temperature in the range of from 155-180° C., more preferably at a temperature in the range of from 155-170° C.

In step (b) at least part of the C₅⁺ hydrocarbons as obtained in step (a) is separated into a light stream and a heavy durene-rich stream. The heavy durene-rich stream as obtained in step (b) suitably contains C₈⁺ aromatics in an amount in the range of from 50-99 wt % preferably 85-95 wt % based on total weight of the heavy durene-rich stream. In the context of the present invention a heavy durene-rich stream is defined as a stream comprising more than 2 wt % of durene, based on total weight of the stream. It will be understood that durene is 1,2,4,5-tetramethylbenzene which is an unwanted by-product in gasoline synthesis because it solidifies at room temperature. The light stream as obtained in step (b) will typically not contain C₁₀⁺ aromatics, whereas the heavy durene-rich stream will typically contain C₁₀⁺ aromatics in an amount in the range of from 5-60 wt %, preferably 25-45 wt %, based on total weight of the heavy durene-rich stream. In the context of the present invention a light stream is defined as a stream comprising less than 2 wt % of durene, based on total weight of the stream.

In step (c) of the process according to the present invention at least part of the heavy durene-rich stream is subjected to a hydrodealkylation treatment to obtain a stream of hydrocarbons having a reduced durene content. The hydrodealkylation is carried out in the presence of hydrogen and at conditions so as to dealkylate alkyl-substituted aromatic hydrocarbons. In this process toluene, mixed xylenes and heavier aromatics are dealkylated to produce benzene, or toluene is transalkylated to produce benzene and mixed xylenes.

The hydrodealkylation treatment in step (c) can be a thermal hydrodealkylation or a catalytic hydrodealkylation. Preferably, use is made of a catalytic hydrodealkylation treatment.

In case use is made in step (c) of a thermal hydrodealkylation treatment, at least part of the heavy durene-rich stream as obtained in step (b) is passed to a thermal hydrodealkylation reactor. Suitably, such a reactor comprises a vertical cylindrical vessel with inlet means in the upper part. It will be understood that in a thermal hydrodealkylation process no use is made of a catalyst. However, some materials used within a thermal hydrodealkylation reactor may display some catalytic activity at the temperatures applied in operation. Suitably, the upper part (50-66%) of the reactor is empty and the remaining lower part of the reactor comprises means for providing plug flow, such as for instance inert vertical baffles or ceramic balls. The thermal hydrodealkylation treatment can suitably be carried out at a temperature in the range of from 580-850° C. and a pressure in the range of from 20-60 bar. The residence time of the heavy durene-rich stream in the reactor will suitably be in the range of from 4-60 seconds.

In case use is made in step (c) of a catalytic hydrodealkylation treatment, at least part of the heavy durene-rich stream as obtained in step (b) is passed to a hydrodealkylation reactor which includes a catalyst. The catalytic hydrodealkylation treatment can suitably be carried out at a temperature in the range of from 480-850° C. and a pressure in the range of from 20-70 bar. Catalysts to be used in hydrodealkylation processes are as such well-known. A suitable catalyst comprises for instance an oxide of a metal of Group VI-B and/or Group VIII of the Periodic Table on a refractory inorganic oxide. Suitable Group VI-B metals include chromium, molybdenum, and tungsten. Suitable Group VIII metals include platinum, nickel, iron, cobalt, rhenium and manganese. Suitable refractory inorganic oxides include for instance alumina, alumina-silica and zirconia. A preferred catalyst comprises chromium composite on a high surface alumina, such as gamma alumina, with the chromia being present in an amount in the range of from 10-20 wt. % of chromium oxide, based on the weight of alumina. The liquid hourly space velocity of the heavy durene-rich stream can suitably be in the range of from 0.5-5.0 h⁻¹. Suitably, in the reactor a hydrogen/hydrocarbon molar ratio of from 5-15 is applied.

In step (d) at least part of the light stream as obtained in step (b) is mixed with at least part of the stream of hydrocarbons having a reduced durene content as obtained in step (c). Preferably, the entire light fraction as obtained in step (b) is mixed with the entire stream of hydrocarbons having a reduced durene content as obtained in step (c).

Preferably, at least part of the mixture obtained in step (d) is passed to an aromatics extraction unit, for instance a Sulfolane extraction unit. In such an aromatics extraction unit zone benzene and other aromatics such as toluene and xylenes are separated from non-aromatics, obtaining an aromatics stream and a non-aromatics stream. Such aromatics extraction units are as such well-known.

Subsequently, at least part of the aromatics stream so obtained can suitably be passed to a fractionation unit, for example a BTX fractionation unit, wherein the various aromatics are separated from each other. In this way, a benzene stream, a toluene stream and a stream of xylenes can for instance be obtained. Such fractionation units are as such well-known.

### Example According to the Invention

### Example 1

The following example has been modelled using PRO/II software as obtained from Invensys Process Systems, Plano, Houston (USA).

A syngas composition comprising 67 vol. % CO and 33 vol. % H₂ is used as feed for a tubular reactor which contains a mixture of a methanol synthesis catalyst and a methanol conversion catalyst. The methanol synthesis catalyst comprises ZnO—Cr₂O₃. The methanol conversion catalyst comprises crystalline iron alumina silicate. The weight ratio of the methanol synthesis catalyst to the methanol conversion catalyst is 2.5. The H₂/CO molar ratio (molar feed ratio) is 0.5. In the reactor the syngas is converted into hydrocarbons using a temperature of 370° C. and a pressure of 60 bar. The conversion of syngas into hydrocarbons is 63% with compounds having 5 and more carbon atoms as 85 wt % of all hydrocarbons produced. The effluent so obtained was passed into a separation unit to obtain a light stream and a heavy durene-rich stream. The light stream comprises 9.1 wt % C₅ paraffins, 12.7 wt % C₆ paraffins, 7.1 wt % C₇ paraffins, 2.0 wt % C₈ paraffins, 2.4 wt % C₉ paraffins, 0.1 wt % C₁₀ paraffins, 0.4 wt % C₅ naphthenes, 1.6 wt % C₆ naphthenes, 4.6 wt % C₇ naphthenes, 5.7 wt % C₈ naphthenes 0.7 wt % C₉ naphthenes, 0.4 wt % C₆ aromatics, 6.0 wt % C₇ aromatics, 31.7 wt % C₈ aromatics and 15.5 wt % C₉ aromatics. The heavy durene-rich stream comprises 1.1 wt % C₉ paraffins, 0.6 wt % C₁₀ paraffins, 0.5 wt % C₁₁₊ paraffins, 0.7 wt % C₈ naphthenes, 2.8 wt % C₉ naphthenes, 0.8 wt % C₁₀ naphthenes, 0.5 wt % C₁₁₊ naphthenes, 8.3 wt % C₈ aromatics, 37.7 wt % C₉ aromatics, 24.6 wt % C₁₀ aromatics excluding durene, 11.8 wt % C₁₁₊ aromatics and 10.6 wt % durene. 100 vol. % of the heavy durene-rich stream so obtained is then subjected to a catalytic hydrodealkyation treatment. The catalytic hydrodealkylation treatment is carried out at a temperature of 550° C. and a pressure of 40 bar, using a catalyst that comprises an oxide of a Group VI-B metal. The product stream so obtained is mixed with the light stream and the mixture then passed to an aromatic extraction unit, and the aromatics stream thus obtained is then passed to a fractionation unit where C₆, C₇ and C₈ aromatics are further separated to a very high degree of purity. In table 1 components of the product so obtained are shown.

### Comparative Example

### Example 2

This example is carried out in the same manner as the Example 1 according to the invention, except that the heavy durene-rich stream was not subjected to the catalytic hydrodealkylation treatment. In table 1 components of the product so obtained are shown.

From the results as shown in Table 1, it will be clear that the present invention provides a highly attractive process for the combined production of gasoline blend components and aromatics, in particularly benzene. In accordance with the present invention not only an improved gasoline is obtained which contains less aromatics, but also much more benzene per amount of gasoline produced at the same time.

TABLE 1

| | Example 1 | | Example 2 | |
|---|---|---|---|---|
| | Aromatics | Gasoline | Aromatics | Gasoline |
| | stream | stream | stream | stream |
| | Stream flowrate (t/d) | | | |
| Component | 1270 | 1000 | 302 | 1000 |
| Stream components | wt % | wt % | wt % | wt % |
| C₅ paraffins | 0.0 | 13.1 | 0.0 | 6.1 |
| C₆ paraffins | 0.0 | 17.9 | 0.0 | 8.5 |
| C₇ paraffins | 0.0 | 10.0 | 0.0 | 4.8 |
| C₈ paraffins | 0.0 | 2.9 | 0.0 | 1.4 |
| C₉ paraffins | 0.0 | 4.7 | 0.0 | 2.3 |
| C₁₀ paraffins | 0.0 | 1.1 | 0.0 | 0.5 |
| C₁₁₊ paraffins | 0.0 | 0.7 | 0.0 | 0.3 |
| C₅ naphthenes | 0.0 | 0.6 | 0.0 | 0.3 |
| C₆ naphthenes | 0.0 | 2.2 | 0.0 | 1.0 |
| C₇ naphthenes | 0.0 | 6.4 | 0.0 | 3.1 |
| C₈ naphthenes | 0.0 | 8.9 | 0.0 | 4.2 |
| C₉ naphthenes | 0.0 | 4.7 | 0.0 | 2.3 |
| C₁₀ naphthenes | 0.0 | 1.1 | 0.0 | 0.5 |
| C₁₁₊ naphthenes | 0.0 | 0.7 | 0.0 | 0.3 |
| C₆ aromatics | 59.0 | 0.0 | 0.9 | 0.0 |
| C₇ aromatics | 6.5 | 0.0 | 13.2 | 0.0 |
| C₈ aromatics | 34.5 | 0.0 | 86.0 | 0.0 |
| C₉ aromatics | 0.0 | 23.2 | 0.0 | 34.4 |
| C₁₀ aromatics | 0.0 | 1.4 | 0.0 | 22.5 |
| C₁₁₊ aromatics | 0.0 | 0.5 | 0.0 | 7.6 |

1. A process for the preparation of hydrocarbons comprising the steps of:
(a) contacting a mixture of carbon monoxide and hydrogen at an elevated temperature and pressure with a mixture of a methanol synthesis catalyst and a methanol conversion catalyst thereby forming C₅⁺ hydrocarbons;
(b) separating at least part of the C₅⁺ hydrocarbons as obtained in step (a) into a light stream and a heavy durene-rich stream;
(c) subjecting at least part of the heavy durene-rich stream to a hydrodealkylation treatment in the presence of hydrogen to obtain a stream of hydrocarbons having a reduced durene content; and
(d) mixing at least part of the light stream as obtained in step (b) with at least part of the stream of hydrocarbons having a reduced durene content as obtained in step (c).

2. A process according to claim 1, wherein the methanol synthesis catalyst comprises a zinc-containing composition which, in addition to zinc, comprises one or more metals chosen from chromium, copper and aluminium.

3. A process according to claim 2, wherein the methanol synthesis catalyst is a ZnO—Cr₂O₃ catalyst.

4. A process according to claim 1, wherein the weight ratio of the methanol synthesis catalyst to the methanol conversion catalyst is in the range of from 0.1-12.5.

5. A process according to claim 1, wherein the methanol conversion catalyst comprises a crystalline (metallo)silicate comprising in addition to SiO₂ one or more oxides of a trivalent metal chosen from aluminium, iron, gallium, rhodium, chromium and scandium, and having a SiO₂/Al₂O₃ molar ratio of at least 10.

6. A process according to claim 5, wherein the methanol conversion catalyst is a crystalline iron alumina silicate or a zeolite selected from the group consisting of ZSM-5, ZSM-11, ZSM-12, ZSM-35 and ZSM-38.

7. A process according to claim 1, wherein step (a) is carried out at a temperature of 200-500° C., a pressure of 1-150 bar and a space velocity of 50-10000 N1.1⁻¹.h⁻¹.

8. A process according to claim 1, wherein the mixture of carbon monoxide and hydrogen in step (a) has a H₂/CO molar ratio in the range of from 0.3-2.

9. A process according to claim 1, wherein the separation in step (b) is carried out at a temperature in the range of from 150-190° C.

10. A process according to claim 1, wherein the heavy durene-rich stream as obtained in step (b) contains C₈⁺ aromatics in an amount in the range of from 50 to 99 wt %, based on total weight of the heavy durene-rich stream.

11. A process according to claim 1, wherein the hydrodealkylation treatment is a catalytic hydrodealkylation treatment.

12. A process according to claim 1, wherein the entire light stream as obtained in step (b) is mixed with the entire stream of hydrocarbons having a reduced durene content as obtained in step (c).
